(19) United States
(12) Patent Application Publication
(10) Pub. No.: US 2026/0364000 A1
(43) Pub. Date: Nov. 19, 2026

(54) VACCINIUM MACROCARPON EXTRACT AND ANTI-AGEING COSMETIC EFFECTS THEREOF

(71) Applicant: SOCIETE INDUSTRIELLE LIMOUSINE D'APPLICATION BIOLOGIQUE, OBJAT (FR)

(72) Inventor: Jean PAUFIQUE, OBJAT (FR)

(21) Appl. No.: 19/737,302

(22) Filed: Jul. 10, 2026

Related U.S. Application Data
(63) Continuation of application No. 18/290,491, filed on May 17, 2022.

(30) Foreign Application Priority Data
May 17, 2021 (FR) FR 2105098

(51) Int. Cl. A61K 8/9789; A61K 8/64; A61Q 19/08

(52) U.S. Cl. CPC A61K 8/9789; A61K 8/645; A61Q 19/08

(57) ABSTRACT

A cosmetic active substance comprising at least one extract of Vaccinium macrocarpon which is a hydrolysate of Vaccinium macrocarpon and the extract comprises at least 80% of peptides by weight of dry matter of the extract, in particular the extract may be an enzymatic hydrolysate; and cosmetic compositions comprising the cosmetic active substance; as well as anti-aging cosmetic uses of the composition and extract.

### CROSS-REFERENCE TO RELATED APPLICATIONS

This application is a continuation application of U.S. patent application Ser. No. 18/290,491 filed Nov. 28, 2023 which is the us national stage application of PCT/EP2022/063237 having the international filing date of May 17, 2022 and claiming the benefit of foreign priority to French patent application FR2105098 filed May 17, 2021, the entire disclosure of these applications is herein incorporated by reference in their entirety.

### TECHNICAL FIELD

The invention relates to a Vaccinium macrocarpon extract comprising at least 80% peptides by weight of dry matter of the extract, in particular to a cosmetic active substance comprising same, to a cosmetic composition comprising said active substance and to anti-aging cosmetic uses thereof.

### BACKGROUND

The growth of videoconferencing has deeply changed our personal and professional communication modes and created new cosmetic concerns, like Zoom Face. This phenomenon occurs when our face appears live and in close-up on the screen and we can't keep from analyzing wrinkles and other defects along with our expressions. Thus, the boom of virtual meetings has caused an explosion of Internet searches on global anti-aging solutions.

The offer of anti-aging ingredients is abundant. Many active molecules are already known for their cosmetic efficiency and their innocuousness. Among these so-called reference molecules, mention may be made of retinol, niacinamide, vitamins C and E, and peptides. These have established themselves as essential ingredients since the 1980s, during which period they began to be integrated into cosmetic products.

Most of the peptides present on the market are of synthetic origin, derived from the chemical industry or biotechnology. Synthetic peptides have an admittedly powerful efficiency, but have the disadvantage of being restricted to a biological target. Although they are described as natural due to their composition in amino acids, their chemical production requires the use of toxic solvents. Synthesis by biotechnology in turn requires the use of genetically modified organisms (GMO). These production methods are no longer in line with consumers' current needs, which place cosmetic care from the botanical world at the top of their expectations.

However, the plant world harbors very diverse molecules, among which natural peptides are noted as highly promising active compounds. Although some peptides freely exist in this form, most are derived from native proteins from which they can be released by hydrolysis.

There is therefore a need for novel cosmetic ingredients of natural origin and consisting of peptides of natural origin for combating skin aging and thus exhibiting overall anti-aging efficacy.

### SUMMARY

To meet this need, the invention proposes a novel extract obtained from cranberry (Vaccinium macrocarpon) comprising at least 80% of peptides by weight of dry matter of the extract; in particular this is a cosmetic active substance comprising said extract for an anti-aging cosmetic application.

Cranberry (Vaccinium macrocarpon (A.)) is an evergreen shrub measuring about thirty centimeters. It grows in the wild in the acidic bogs of the cold regions, mountainous forests and sandy prairies of North America. In the summer, bell-shaped pink and purple flowers offer their open corolla to pollinators. Over the course of the season, the development of the red berries transforms these regions into vast vibrant areas. The mature fruit is collected in the fall.

Cranberry, also called Atoca by Native Americans, was traditionally used for its medicinal properties. When the first Europeans arrived in the US territory, the colonists in turn discovered and appreciated this tangy berry. Beginning in the mid-19^{th} century, German doctors contributed to the spread of its consumption in the modern world. Cranberry has gradually become the health food of our time and today is considered to be a true superfruit.

Cranberry is in particular well known for its nutritional importance and its health benefits, in particular for its antioxidant effects. In fact, cranberry is rich in vitamin C and other antioxidants to prevent the appearance of certain cancers, cardiovascular diseases, and various aging-related illnesses. Cranberry is also used in the prevention of urinary tract infections, as well as to treat or prevent gingivitis and periodontal disease.

Finally, anti-aging qualities have been attributed to it due to the presence of numerous antioxidant molecules.

The present invention relates to a specific peptide-rich extract lacking the antioxidant molecules naturally present in the fruit, and having a cosmetic efficacy, in particular anti-aging.

Thus, the invention relates to an extract of Vaccinium macrocarpon comprising at least 80% of peptides by weight of dry matter of the extract.

Preferentially, said extract is obtained from the fruit of Vaccinium macrocarpon, more preferentially from cakes of Vaccinium macrocarpon.

Said extract is a cosmetic active substance particularly of interest as natural ingredient for combating skin aging.

Thus, the cosmetic active substance according to the invention comprising the peptides of natural origin advantageously has an overall anti-aging effect; in particular, it has an anti-wrinkle effect and/or allows improved complexion radiance and/or improved quality of the dermal-epidermal junction, and/or improved skin homeostasis.

It therefore responds to the problems of the prior art, and the present invention targets the cosmetic applications cited above, preferentially when the active substance is integrated into a cosmetic composition comprising at least 0.1% of said active substance and a physiologically acceptable medium, for topical application.

The present invention also relates to a method for obtaining the active substance according to the invention comprising in particular a solubilization of the fruit cakes of Vaccinium macrocarpon in water, followed by enzymatic hydrolyses, separation of the soluble and insoluble phase and recovery of the soluble phase, enzymatic inactivation, purification of the peptide fraction and optionally concentration of the extract followed by sterilizing filtration.

Other features and advantages will emerge from the detailed description of the invention and the following examples.

### DETAILED DESCRIPTION

### Definition

Within the meaning of the invention, “cosmetic active substance” means an extract comprising at least one molecule, preferentially a set of molecules, more preferentially the molecules are peptides, having a cosmetic effect on the skin. Preferentially, the cosmetic effect on the skin is an anti-aging effect.

Within the meaning of the invention, “Vaccinium macrocarpon” refers to a shrub of the family Ericaceae, also known as Large cranberry, American cranberry, Cranberry, or under the scientific names: Oxycoca macrocarpa (A.), Oxycoccus macrocarpus (A.) or Schollera macrocarpos (A.).

Within the meaning of the invention, “hydrolysate of “Vaccinium macrocarpon” means any active substance derived from fruit of Vaccinium macrocarpon, in particular cakes of Vaccinium macrocarpon, obtained by a method comprising at least one hydrolysis step, preferentially two successive hydrolysis steps of Vaccinium macrocarpon. The term “hydrolysate of Vaccinium macrocarpon” excludes the molecules extracted only by maceration or decantation of Vaccinium macrocarpon.

Within the meaning of the invention, the term “peptides” refers to peptides of size, expressed in molar mass, less than or equal to 2,000 Da.

Within the meaning of the invention, the term “acidic amino acids” refers to a negatively charged amino acid at neutral pH, such as aspartic acid or glutamic acid.

Within the meaning of the invention, the term “basic amino acids” refers to a positively charged amino acid at neutral pH, such arginine, histidine or lysine.

Within the meaning of the invention, the term “cationic peptides” refers to peptides of size, expressed in molar mass, less than or equal to 2,000 Da and formed predominantly of basic amino acids.

Within the meaning of the invention, the term “cakes” refers to the solid residues resulting from the extraction of the oil from the seeds or fruits, in particular seeds and pulp of Vaccinium macrocarpon. The seeds and the pulp are in particular ground and the oil is extracted by a cold press extraction method well known to a person skilled in the art. This method allows recovery of the cranberry oil and solid residual co-products: the cakes.

“Film,” within the meaning of the invention, is understood to mean a cosmetic product that is applied momentarily to the skin and removed after a certain application time, and that exhibits a cosmetic or dermocosmetic effect. It may, for example, be a mask, a patch or a strip, for the face or the body.

### Active Substance According to the Invention

The present invention therefore relates to a cosmetic active substance comprising at least one extract of Vaccinium macrocarpon comprising at least 80% of peptides by weight of dry matter of the extract.

The extract according to the invention is an ingredient of natural origin, including the peptides present in said extract. The specificity of the extract according to the invention is that it consists of at least 80% of peptides of natural origin, that is, they are not synthetic and therefore derived from the chemical industry or biotechnology. To do this, the extract is obtained from Vaccinium macrocarpon, preferentially fruit, more preferentially cakes of Vaccinium macrocarpon.

The cakes of Vaccinium macrocarpon are preferentially obtained after a first step of extracting cranberry fruit, allowing collection on the one hand of cranberry juice and on the other hand of seeds and residual pulp derived from the fruit. The seeds and the pulp are then ground and the oil extracted by cold pressing. The cranberry cake then remains, which is the raw material of the extract according to the invention.

According to a preferred embodiment, the extract according to the invention is a hydrolysate of Vaccinium macrocarpon, preferentially an enzymatic hydrolysate of the cakes of Vaccinium macrocarpon.

Particularly advantageously, the enzymatic hydrolysate is obtained after two successive enzymatic hydrolyses carried out by two different proteases.

The extract according to the invention then preferentially comprises at least 80% of peptides by weight of dry matter of the extract. According to a particularly preferred embodiment, the peptides represent 98% by weight of dry matter of the extract. Said peptides have a size, expressed in molar mass, of less than or equal to 2,000 Da. Preferentially, said peptides have a size of between 243 Da and 2,000 Da.

The distribution and the quantity of the peptides can be determined by a spectrophotometric assay according to the Lowry method (Lowry et al., Protein measurement with the Folin reagent, J. Biol. Chem., 193, 265-275, 1951). The characterization and quantification of the amino acids present in the extract and therefore in the active substance according to the invention can be carried out by liquid chromatography after total hydrolysis of the sample.

Thus, the extract according to the invention is preferentially and predominantly composed of peptides comprising between 4 and 6 amino acids, said peptides being enriched in basic amino acids, preferentially at least 20%, more preferentially at least 25%, even more preferentially 27.9%, particularly in arginine, preferentially at least 15%, more preferentially 19.6%.

Thus, the extract according to the invention has a content of cationic peptides, said peptides being formed predominantly of basic amino acids, greater than 20%.

The inventor also determined the dry matter content, the ash content and the total sugar content of the extract.

The content of dry matter can be determined by weighing the residues resulting from the drying of the samples of the extract according to the invention at 105 °C in an oven until a constant weight is obtained.

The mineral ash content can be determined by weighing the residues from the incineration of the samples of the active substance according to the invention at 550 °C in an electric muffle furnace.

The sugar content (carbohydrates) in the active substance comprising said extract can be determined using the DUBOIS method (Dubois M. et al., Analytical chemistry, 28, 3, 350-356, 1956). The total sugar content of the active substance is expressed as a percentage relative to the dry material.

The assay of the total nitrogen in the extract according to the invention can be carried out in particular by the KJELDHAL method (reference: Official method of analysis of the A.O.C., 12th ed. W Horwitz, E.D., New-York, 15-60, 1975). The content is expressed as a percentage relative to the dry matter content.

The extract according to the invention preferentially comprises 95% of nitrogenous fraction (or the peptides) and 5% of ash, by weight relative to the dry matter. It therefore does not comprise sugars.

The active substance according to the invention comprises the extract alone or with other constituents.

The cosmetic active substance according to the invention can be in liquid form or in solid form or in film form.

When it is in liquid form, the active substance according to the invention is preferentially exclusively composed of the extract of Vaccinium macrocarpon cakes accompanied by stabilizers and/or preservatives.

The extract in liquid form is preferentially in the form of a clear liquid aqueous solution, with a low odor and a very light yellow color. However, it may be more colored.

Preferentially, the extract according to the invention in liquid form has a dry matter content of: 5 g/L to 30 g/L, even more preferentially from 9 g/L to 14 g/L.

When the active substance according to the invention is in solid form, it is preferentially in powder form with or without support. The support may be, for example, chosen from maltodextrins. In this case, preferentially, the extract represents at least 5% by weight and the support at most 95% by weight.

The active substance according to the invention can also be presented in the form of a film. In this case, the extract of Vaccinium macrocarpon cakes preferentially represents at least 0.1% by weight of the film.

When it is in the form of a film, the active substance comprises:
at least the hydrolysate of cakes of Vaccinium macrocarpon according to the invention.
at least one mineral filler, and
at least one polymer of natural origin, and
at least one plasticizer, and
at least one surfactant, and

The polymer of natural origin may be chosen from: Pectin, tamarind gum, alginate, pullulan, psyllium, xanthan, guar, tara, carob, agar, gum arabic, gellan, dextran, carrageenan, cellulose, konjac and chitosan.

The plasticizer may be chosen from: Glycerol, sorbitol, saccharose, erythritol, urea, propylene glycol and butylene glycol.

The mineral filler may be chosen from: Calcium carbonate, green clay, kaolin, perlite, talc, magnesium silicate, mica, diatomaceous sericite, silica, calcium sulfate, calcium chloride, potassium chloride, iron oxide and zinc oxide.

The active substance may also comprise a pigment for coloring the film.

According to a preferred embodiment, the extract of the cosmetic active substance according to the invention is capable of being obtained by an extraction method comprising at least one enzymatic hydrolysis of cakes of Vaccinium macrocarpon, the cakes having been solubilized beforehand in water.

According to a particularly preferred embodiment, the extract of the cosmetic active substance according to the invention is capable of being obtained by an extraction method comprising at least two enzymatic hydrolyses of cakes of Vaccinium macrocarpon, the cakes having been solubilized beforehand in water.

Preferentially, the two successive enzymatic hydrolyses are carried out with two different proteases. The method preferentially next comprises sorting of the molecules present in the soluble phase selected after hydrolysis to purify the peptide fraction, and particularly the peptides with sizes of less than 2,000 Da.

Thus, the extract of the cosmetic active substance according to the invention is capable of being obtained by the following method:
solubilization of at least 100 g/L of Vaccinium Macrocarpon cakes in water,
enzymatic hydrolysis,
purification of the peptide fraction.

According to a particularly preferred embodiment, the extract of the active substance according to the invention is capable of being obtained by a method as described hereinafter.

### Method for Obtaining the Extract According to the Invention

The extract according to the invention can be obtained by any means.

According to a particularly suitable embodiment, the extract according to the invention is obtained by implementing the following steps:
Solubilization of fruit from Vaccinium macrocarponin water, preferentially cakes of Vaccinium macrocarpon, preferentially at least 100 g of the cakes of Vaccinium macrocarpon per liter of water,
at least two successive enzymatic hydrolyses, preferentially at least two enzymatic hydrolyses carried out using at least two different proteases,
separating the soluble and insoluble phases by any means making it possible to separate an insoluble phase from the soluble phase, for example by centrifugation, filtration or decantation,
recovering the soluble phase,
enzymatic inactivation by any means allowing the inactivation of the enzymatic activities, for example by heat treatment,
molecular sorting of the molecules of the soluble phase in order to recover the molecules of size less than 2,000 Da and in particular peptides with sizes of less than 2,000 Da, preferentially of size between 243 Da and 2,000 Da, by any means allowing molecular sorting, for example by ultrafiltration or ion chromatography,
optionally deodorization or bleaching,
preferentially filtering and sterilizing filtration.

During the extraction of cranberry juice, fruit seeds and pulp remain. Secondly, during the extraction of the oil by cold pressing, only the cakes remain. This process is usual in the food processing industry. These co-products are then reduced to powder. Thus, only the molecules of the fruit, preferentially cakes, constitute the starting raw material.

Molecular sorting can be carried out by ultrafiltration; preferentially, molecular sorting is carried out by ion chromatography.

An additional step for drying the hydrolysate can be added so that it is in solid form, preferentially in powder form. It may be an atomization or freeze-drying step, for example. In this case, a support such as for example a maltodextrin can be used.

The steps of the methods described above, taken individually are common in the field of extraction of active substances from natural raw materials and the person skilled in the art is able to adjust the reaction parameters on the basis of his general knowledge, and so as to obtain the characteristics of the active substance which is the object of the invention.

### Cosmetic Composition

The active substance according to the invention is preferably used in cosmetic compositions comprising a cosmetically acceptable medium, preferentially a cosmetically acceptable medium. The compositions are in different galenical forms, suitable for topical application to the skin.

These compositions may in particular be in the form of oil-in-water emulsions, water-in-oil emulsions, multiple emulsions (Water/Oil/Water or Oil/Water/Oil) which may optionally be microemulsions or nanoemulsions, or in the form of solutions, suspensions, hydrodispersions, aqueous gels, powders, or foundation. They may be more or less fluid and have the appearance of creams, emulsions, gels, masks or any other aspects of healthy skin care cosmetics.

They can be compositions comprising at least 0.1% of the liquid active substance according to the invention (or about 7.2 ppm of peptides), preferentially between 0.5 and 10% or comprising at least 0.01% of a solid active substance according to the invention.

In addition to the active substance, these compositions comprise a physiologically acceptable and preferably cosmetically acceptable medium, that is, one that does not cause sensations of discomfort to the user, such as redness, tightness, or tingling.

As an additive, the compositions according to the invention may contain at least one compound selected from:
oils, which may be chosen in particular from linear or cyclic, volatile or non-volatile silicone oils,
waxes, such as ozokerite, polyethylene wax, beeswax or carnauba wax,
silicone elastomers,
surfactants, preferably emulsifiers, whether non-ionic, anionic, cationic or amphoteric,
co-surfactants, such as linear fatty alcohols,
thickeners and/or gelling agents,
humectants, such as polyols such as glycerin,
dyes, preservatives, fillers,
tighteners,
sequestrants,
perfumes,
and mixtures thereof, without this list being exhaustive.

Examples of such additives are cited in particular in the CTFA Dictionary (International Cosmetic Ingredient Dictionary and Handbook published by the Personal Care Product Council).

Of course, a person skilled in the art will take care to choose any additional, active or non-active compounds, and their quantity, such that the advantageous properties of the mixture are not, or substantially not, altered by the envisaged additive.

These compositions are intended to be used on healthy skin, in particular mature skin, in particular for an overall anti-aging effect, preferentially an anti-wrinkle effect and/or to improve complexion radiance and/or to improve the quality of the dermal-epidermal junction and/or to improve skin homeostasis.

The invention therefore also relates to a cosmetic method for treating the skin for an anti-wrinkle effect and/or to improve the complexion radiance and/or to improve the quality of the dermal-epidermal junction, and/or to improve skin homeostasis which consists in topical application to the skin of a healthy person of such an extract according to the invention or of such a composition according to the invention.

### Use

The active substance according to the invention thus has characteristics that allow its use in cosmetics and in particular to limit the natural effects related to aging and thus to obtain an overall anti-aging effect, preferentially an anti-wrinkle effect and/or to improve the complexion radiance and/or to improve the quality of the dermal-epidermal junction and/or to improve skin homeostasis.

It thus allows improved homeostasis of the skin and therefore the barrier function in elderly healthy subjects, which is reflected directly by a reduction in transepidermal water losses of 15%. The radiance is also increased by 14%, which results in a renewed complexion radiance. The epidermal barrier function of older skin is therefore reinforced.

The extract according to the invention also promotes the expression of major structural biomarkers of the dermis, such as collagens I and III; periostin, tropoelastin, proteoglycans (decorin, perlecan, biglycan, agrigan); hyaluronic acid.

The extract according to the invention also allows decreased expression of dermis degradation markers such as MMP-1.

Thus, the quality of the dermal fibers is improved by 17%, which results in a reduction in the crow's feet wrinkles in Caucasian and Asian volunteers.

By acting on these various markers, the active substance according to the invention has an overall anti-aging action. Therefore, the beauty and quality of the skin are sublimated, wrinkles are significantly reduced, the skin is firmer and the complexion radiance is renewed. It is particularly suited to healthy mature skin.

The active substance comprising at least one extract of the cakes of Vaccinium macrocarpon can thus be used for cosmetic, and therefore by definition non-therapeutic, anti-aging uses in topical application to healthy skin.

The active substance differs from cranberry extracts commonly known for their anti-oxidant effect since it does not contain polyphenols giving it this efficacy.

The invention is now illustrated by non-limiting examples of compositions according to the invention and by results of tests.

### EXAMPLES

### Example 1: Extract of Vaccinium macrocarpon without the Extraction Method According to the Invention (Outside the Invention)

The characterization and quantification of the constituent amino acids of a Cranberry cake were carried out by liquid chromatography.

TABLE 1

| Amino acid name | Distribution (%) |
|---|---|
| Alanine | 5.0 |
| Arginine | 12.2 |
| Aspartic acid | 10.8 |
| Glutamic acid | 22.3 |
| Cysteine | 0.0 |
| Glycine | 5.8 |
| Histidine | 2.2 |
| Isoleucine | 4.3 |
| Leucine | 7.9 |
| Lysine | 2.9 |
| Phenylalanine | 5.0 |
| Proline | 2.9 |
| Serine | 5.0 |
| Threonine | 3.6 |
| Tyrosine | 3.6 |

The cranberry cake has a basic amino acid content (arginine, histidine, lysine) of 17.3% and an acidic amino acid content (aspartic acid, glutamic acid) of 33.1%.

### Example 2: Extract According to the Invention

The active ingredient is obtained by implementing the following steps:
solubilization in water of the cakes of Vaccinium macrocarpon at a rate of 100 g/L,
enzymatic hydrolysis,
enzymatic inactivation by heat treatment,
step of molecular sorting by ion chromatography, intended to select the molecules, in particular the peptides, of sizes of less than 2,000 Da,
deodorization
filtration and sterilizing filtration.

The obtained active ingredient has the following analytical characteristics:
a dry matter content: 12.2 g/L
a protein content: 10.2 g/L (83.6%)
a pH: 3.0-4.0
ash level: 0.6 g/L (4.9%).

The characterization and quantification of the constituent amino acids of the extract according to the invention were carried out by liquid chromatography after hydrolysis of the sample.

The distribution of the various amino acids is shown in Table 2

TABLE 2

| Amino acid name | Distribution (%) |
|---|---|
| Alanine | 6.2 |
| Arginine | 19.6 |
| Aspartic acid | 10.3 |
| Glutamic acid | 20.6 |
| Cysteine | 0.0 |
| Glycine | 5.2 |
| Histidine | 3.1 |
| Isoleucine | 4.1 |
| Leucine | 8.2 |
| Lysine | 5.2 |
| Phenylalanine | 2.1 |
| Proline | 0.0 |
| Serine | 5.2 |
| Threonine | 3.1 |
| Tyrosine | 3.1 |

The extract according to the invention has a higher basic amino acid content (27.9%) relative to the cranberry cake of example 1 due to a significant arginine content and a lower acidic amino acid content (30.9%) due to a decrease in the aspartic acid content. The steps of the method according to the invention allow selection of the cationic peptides (rich in basic amino acids: arginine, histidine, lysine).

The active substance according to the invention does not have the characteristic often claimed by cranberry extracts, namely the active substance according to the invention does not have a free-radical effect.

Indeed, the extract according to the invention does not have anti-radical activity according to the DPPH test as shown in Table 3 below.

Diphenyl-picrylhydrazyl hydrate (DPPH) is a free radical, absorbent in the violet range at 517 nm. An anti-radical product causes the violet coloration to disappear. The anti-radical activity of a substance is expressed as a percentage of inhibition of the absorbance of DPPH.

TABLE 3

| | Percentage of inhibition of |
|---|---|
| | the absorbance of DPPH |
| Pure active substance | 10% |
| 50% of active substance | 7% |
| 10% of active substance | 3% |

At the recommended maximum dose, that is, 10% of active substance, the percentage of inhibition of the absorbance of the DPPH is low. Thus, the extract according to the invention does not have anti-radical activity.

### Example 3: Comparative Example with Extracts Outside the Invention (OI)

The following products are obtained from the cakes of Vaccinium macrocarpon with a method different from the method of example 2.

Product OI 1 is obtained with a method which differs from the method of example 2 in that it does not contain a hydrolysis step.

The product obtained has the following analytical characteristics:
A dry matter content of 26.7 g/L
A protein content of 1.6 g/L (6%)
A sugar content of 11.4 g/L (57%)

The product therefore has a protein content of 6% relative to the dry matter content and therefore much lower than the minimum content of 80% claimed in the context of the invention.

Product OI 2 is obtained with a method which differs from the method of example 2 in that it does not contain the molecular sorting steps.

The product obtained has the following analytical characteristics:
A dry matter content of 53.8 g/L
A protein content of 19 g/L (35%)
A sugar content of 17.3 g/L (32%)

The product has a protein content of 35% relative to the dry matter content and much lower than the minimum content of 80% claimed in the context of the invention.

The efficacy of the OI 2 extract and the efficacy of the present invention were studied by following the expression of genes linked to the barrier function and to the matrix dynamics, according to the protocol described below. The comparative results between the extract outside the invention and the extract of the present invention are presented in table 4 below.

TABLE 4

| | | Extract derived from Example 2 | OI 2 |
|---|---|---|---|
| On older | Ki67 | + | + |
| keratinocytes | Claudin1 | + | + |
| | IL8 | ++ | − |
| | loricrin | + | − |
| On older | MMP1 | + | + |
| fibroblasts | BGN | + | − |
| | AQP3 | + | − |

It clearly appears that the OI 2 extract derived from a different method and containing less than 80% of peptides does not exhibit an efficacy similar to that of the extract according to the invention for the expression of the genes studied.

### Example 4: Example of a Composition According to the Invention

An example of a formulation comprising the active substance according to the invention in creamy gel form is presented in table 5 below:

TABLE 5

| | Ingredients | % |
|---|---|---|
| A | Water | q.s. 100 |
| | Glycerin | 2.00 |
| | Butylene Glycol | 3.00 |
| B | Carbomer | 0.30 |
| C | Aqua (Water) & Sodium Hydroxide | 0.20 |
| D | Caprylyl Methicone | 3.00 |
| | Dimethicone | 3.00 |
| | Dimethicone & Dimethicone/Vinyl | 5.00 |
| | Dimethicone Crosspolymer | |
| E | Sodium Acryloyldimethyltaurate/VP | 0.75 |
| | Crosspolymer | |
| F | Preservative | qs |
| G | Invention active substance | 2.00 |
| H | Aqua (Water) & Sodium Hydroxide | q.s. pH 7.0-7.5 |

The composition of example 4 can in particular be obtained by the following method:
Disperse B in A under gentle stirring.
Pre-neutralization with C.
Under vigorous stirring, add D then E.
Maintain the stirring for 20 min.
Under moderate stirring, add F and G.
Adjust the pH with H.

The composition is then in the form of a thick and shiny, white, broken creamy gel having a pH at 1 month equal to 7.3, a viscosity (C/5 rpm) equal to 44,200 cP.

### Example 5: Example of a Composition According to the Invention

An example of a formulation comprising an active substance according to the invention in unctuous cream form is presented in table 6 below:

TABLE 6

| | Ingredients | % |
|---|---|---|
| A1 | Water | q.s. 100 |
| | Monosodium Citrate | 0.20 |
| | Preservative | qs |
| | Erythritol | 2.00 |
| A2 | Butylene Glycol | 3.00 |
| | Xanthan Gum | 0.20 |
| B | PEG-100 Stearate & Glyceryl Stearate | 3.00 |
| | Arachidyl Alcohol & Behenyl Alcohol & | 3.00 |
| | Arachidyl Glucoside | |
| | Pentaerythrityl Distearate | 0.50 |
| | Butyrospermum parkii (shea) butter | 2.00 |
| | Isocetyl Stearate | 7.00 |
| | Cetearyl ethylhexanoate | 8.00 |
| | Diisopropyl Sebacate | 4.00 |
| | Dimethicone | 3.00 |
| | Argania Spinosa Kernel Oil | 3.00 |
| | Tocopherol & Helianthus Annuus (Sunflower) | 0.10 |
| | Seed Oil | |
| C | Hydroxyethyl Acrylate/Sodium Acryloyldimethyl | 0.50 |
| | Taurate Copolymer | |
| D | Propylene glycol | 2.00 |
| | Alumina & CI 77891 (Titanium Dioxide) | 1.00 |
| E | Active substance according to the invention | 2.00 |
| F | Aqua (Water) & Sodium Hydroxide | q.s. pH |
| | | 5.0-6.0 |

The composition of example 5 can in particular be obtained by the following method:
Add A2 into A1 under moderate stirring, stir until a homogeneous gel is obtained and heat to 80 °C.
Place B under magnetic stirring and heat to 80 °C
Emulsify B in A with A rotor-stator for 10 min.
At 50 °C, under moderate stirring, add C then D and E.
Cool down to room temperature under gentle stirring, then adjust the pH with F.

The composition is then in the form of a thick, white and shiny emulsion having a pH at 1 month equal to 6.0, a viscosity (C/5 rpm) equal to 57,000 cP.

### Example 6: Example of a Composition According to the Invention

An example of a formulation comprising the active substance according to the invention in night cream form is presented in table 7 below:

TABLE 7

| | Ingredients | % |
|---|---|---|
| A1 | Water | q.s. |
| | | 100 |
| | Sodium Citrate | 0.20 |
| | Preservative | qs |
| A2 | Isopentyldiol | 2.00 |
| | Glycerin | 1.50 |
| | Xanthan Gum | 0.10 |
| B | Cetyl Alcohol & Glyceryl Stearate & PEG-75 | 4.00 |
| | Stearate & Ceteth-20 & Steareth-20 | |
| | Potassium cetyl phosphate | 0.75 |
| | Cetearyl Alcohol | 1.00 |
| | Hydrogenated Coconut Oil | 2.00 |
| | Pentaerythrityl Tetraisostearate | 3.00 |
| | Triheptanoin | 3.00 |
| | Isopropyl Isostearate | 6.00 |
| | Isopropyl Palmitate | 6.00 |
| | Dimethicone | 3.00 |
| | Tocopherol & Helianthus Annuus (Sunflower) Seed Oil | 0.05 |
| C | Hydroxyethyl Acrylate/Sodium Acryloyldimethyl | 1.00 |
| | Taurate Copolymer & Polyisobutene & Aqua (Water) & | |
| | PEG-7 Trimethylolpropane Coconut Ether & Sorbitan | |
| | Isostearate | |
| D | Active Substance According to the Invention | 2.00 |

The composition of example 6 can in particular be obtained by the following method:
Add A2 into A1 under moderate stirring, stir until a homogeneous gel is obtained and heat to 80 °C.
Place B under magnetic stirring and heat to 80 °C
Emulsify B in A under shearing stirring for 10 minutes.
At 40-50 °C, under moderate stirring, add C then D.

The composition is then in the form of a thick white and shiny emulsion.

### Example 7: Example of a Composition According to the Invention

An example of a formulation comprising an active substance according to the invention in foundation form is presented in table 8 below:

TABLE 8

| | Ingredients | % |
|---|---|---|
| A | Polyglyceryl-3 Polyricinoleate & Polyglyceryl-3 | 5.00 |
| | Diisostearate | |
| | Hydrogenated Castor Oil | 0.50 |
| | Cera Alba (Beeswax) | 0.50 |
| | Dicaprylyl Carbonate & Stearalkonium Hectorite & | 6.00 |
| | Propylene Carbonate | |
| | Isononyl Isononanoate | 4.00 |
| | Dimethicone | 4.00 |
| | Triheptanoin | 4.00 |
| | C12-15 alkyl benzoate | 4.00 |
| | Magnesium Stearate | 1.00 |
| B | Cocoglycerides | 12.00 |
| | CI 77499 (Iron Oxides) & Hydrogenated Lecithin | 0.10 |
| | CI 77491 (Iron Oxides) & CI 77499 (Iron Oxides) & | 0.50 |
| | Hydrogenated Lecithin | |
| | CI 77891 (Titanium Dioxide) & Hydrogenated Lecithin | 8.00 |
| | CI 77492 (Iron Oxides) & Hydrogenated Lecithin | 1.00 |
| C | Aqua (Water) | q.s. 100 |
| | Glycerin | 5.00 |
| | Magnesium Sulfate | 1.00 |
| | Preservative | qs |
| | Polyglyceryl-3 Polyricinoleate & Polyglyceryl-3 | |
| | Diisostearate | |
| D | Active substance according to the invention | 2.00 |

The composition of example 7 can in particular be obtained by the following method:
Heat A at 85 °C and homogenize with vigorous stirring.
Grind B in the triple-roller and transfer to A. Stir until homogenized.
Place C under magnetic stirring and heat to 80 °C
Add C in AB very slowly under vigorous stirring.
Homogenize for 10 min under shearing stirring.
At 30 °C, add D and homogenize for 2 min under shearing stirring.

The composition is then in the form of a supple and shiny emulsion.

### Effectiveness Tests

### Test 1: Evaluation of the Activity Potential of the Active Substance According to the Invention on the Major Genes of Skin Homeostasis

The objective of this study is to evaluate the activity potential of the active substance according to the invention on the major genes involved in skin equilibrium.

This study was carried out on 34 genes involved in the various processes related to the establishment of:
the skin barrier function: proliferation, hydration, cohesion, differentiation, desquamation, innate immunity, anti-oxidant and inflammation system (cf. Table 9: Proliferation, Hydration, Cohesion, Differentiation, Anti-Radical and Inflammation).
the dermal-epidermal junction DEJ;
the dermal matrix; organization and degradation of the fibers, hydration and inflammation (cf. Table 9: Matrix Organization, Matrix Degradation, Hydration, Proteoglycan, Growth and Inflammation Factors).

The expression of the various targeted genes was evaluated by quantitative PCR on human keratinocytes from young donors (≤30 years) and older donors (≥60 years) or young human fibroblasts (<P5) and older human fibroblasts by successive replications (more than 20 replicates).

The procedure of the study is described below. The young and older human cells are seeded and incubated at 37 °C. Then, the cells are treated for 48 h with the active substance according to the invention or a solution of retinol at 0.5.10-5M.

After several days, the cells are recovered and the total RNA extracted. The RNAs were reverse-transcribed and the complementary DNAs obtained were analyzed by the quantitative PCR technique. The mRNAs of internal reference controls were analyzed in parallel with the mRNAs of the markers involved in the barrier function of the epidermis, in the DEJ and in the dermal matrix.

The incorporation of fluorescence is measured continuously using a thermal cycler. The RQ (relative quantification) analysis is performed using software.

The results are shown in table 9.

TABLE 9

| | | | Older cells | | | | |
|---|---|---|---|---|---|---|---|
| | | Young | | Active | Efficacy of | | Efficacy of |
| | | Cells | | substance of | the PA/Older | 1 μM | retinol/older |
| | Genes | Control | Control | example 2 0.5% | control (%) | Retinol | control (%) |
| Proliferation | MKI67 | 100 | 66 | 93 | +41 | 95 | +44 |
| Hydration | AQP3 | 100 | 61 | 84 | +38 | 69 | +13 |
| | HAS3 | 100 | 68 | 101 | +49 | 357 | +425 |
| | CD44 | 100 | 83 | 104 | +25 | 147 | +77 |
| Cohesion | CLDN1 | 100 | 69 | 90 | +30 | 33 | −52 |
| | DSG | 100 | 72 | 85 | +18 | 1 | −99 |
| Differentiation | FLG | 100 | 38 | 56 | +47 | 9 | −76 |
| | LOR | 100 | 20 | 35 | +75 | 1 | −95 |
| | TGM1 | 100 | 68 | 89 | +31 | 47 | −31 |
| | KRT10 | 100 | 73 | 91 | +25 | 2 | −97 |
| | IVL | 100 | 53 | 64 | +21 | 120 | +126 |
| | S100A7 | 100 | 38 | 50 | +32 | 564 | +1384 |
| Anti-Radical | SOD1 | 100 | 89 | 100 | +12 | 95 | +7 |
| Inflammation | CXCL8 | 100 | 252 | 161 | −36 | 729 | +189 |
| | S100A8 | 100 | 208 | 186 | −11 | 256 | +23 |
| | S100A9 | 100 | 191 | 177 | −7 | 397 | +108 |
| Structural | LAMA3 | 100 | 103 | 114 | +11 | 94 | −9 |
| elements | COL4A1 | 100 | 40 | 54 | +35 | 38 | −5 |
| of the DEJ | COL7A1 | 100 | 86 | 105 | +22 | 68 | −21 |
| | COL17A1 | 100 | 97 | 113 | +16 | 81 | −16 |
| Matrix | COL1A1 | 100 | 63 | 78 | +24 | 79 | +25 |
| Organization | COL3A1 | 100 | 43 | 53 | +23 | 72 | +67 |
| | POSTN | 100 | 45 | 43 | −4 | 56 | +24 |
| | ELN | 100 | 60 | 77 | +28 | 63 | +5 |
| | FBN1 | 100 | 91 | 99 | +9 | 90 | −1 |
| Matrix | MMP1 | 100 | 216 | 136 | −37 | 164 | −24 |
| Degradation | | | | | | | |
| Hydration | AQP3 | 100 | 56 | 98 | +75 | 73 | +30 |
| Proteoglycan | HSPG2 | 100 | 87 | 87 | 0 | 90 | +3 |
| | BGN | 100 | 63 | 77 | +22 | 78 | +24 |
| | DCN | 100 | 83 | 91 | +10 | 89 | +7 |
| | ACAN | 100 | 38 | 45 | +18 | 10 | −74 |
| | LUM | 100 | 90 | 94 | +4 | 87 | −3 |
| Growth | VEGFA | 100 | 91 | 88 | −3 | 93 | +2 |
| Factors | TGFβ1 | 100 | 95 | 97 | +2 | 103 | +8 |
| Inflammation | CXCL8 | 100 | 210 | 185 | −12 | 803 | +282 |
| | IL1 | 100 | 226 | 160 | −29 | 567 | +151 |

Of the 34 genes evaluated, 76% exhibit modified expression during aging reflecting a major impact on the barrier function, the dermal-epidermal junction and the dermal matrix.

Tested at 0.5% on aged keratinocytes, the active substance according to the invention significantly increases the expression of key genes by:
proliferation (Ki67: +41%);
differentiation (loricrin: +75% and filaggrin; +47%);
cohesion (claudin-1: +30%);
anti-radical defense (SOD1: +12%).

Tested at 0.5% on older fibroblasts, the substance according to the invention acts significantly on the expression of genes associated with matrix organization (collagen 1: +24% and elastin: +28%) and its degradation (MMP1:-37%). On both cell types, significant action was demonstrated on the markers linked to hydration and inflammation. The active substance according to the invention and retinol have similar effects on the genes linked to the matrix.

### Test 2: Biological Efficacy of the Active Substance According to the Invention on the Barrier Function of the Epidermis and Hydration.

The objective of this study is to evaluate the ability of the active substance according to the invention to maintain a functional barrier function by stimulating the synthesis of Ki 67, loricrin, claudin-1 and aquaporin 3.

One of the major strategies of the skin to protect is to maintain an intact barrier. This barrier, resulting from the process of proliferation and differentiation of the keratinocytes, results from the particular structure of the stratum corneum and the cohesion of the upper layers of the epidermis ensured by the tight junctions. Several protein components are essential for the establishment of this barrier function, such as:
Ki-67, which is a key marker for the proliferation of the cells of the basal layer of the epidermis.
Loricrin, which plays a major role in terminal differentiation.
Claudin-1, which is one of the main constituents of tight junctions, complex structures that establish true cell-cell cohesion.
Aquaporin 3, which is part of the membrane proteins essential for transporting water and allows maintained hydration of the stratum corneum.

Proliferation, cohesion and hydration level are thus directly linked to the epidermal differentiation process and to the state of the barrier function.

This study was carried out on normal and older reconstructed epidermises. The epidermal construction was evaluated using a Hematoxylin Eosin (HE) stain and the synthesis of the barrier function markers by immunohistofluorescence.

The procedure of the study is described below. Young and aged human keratinocytes are seeded on inserts and then incubated at 37 °C in an atmosphere containing 5% CO₂ and cultivated for several days.

The reconstructed epidermises are treated for 48 hours systemically with:
The active substance according to the invention at 0.1% and 0.5% (V/V)
A retinol solution at 10 μM.

The epidermides are recovered, fixed, dehydrated, and enclosed in paraffin. Sections (4 μm) are then made using a microtome.

The analysis of the thickness of the reconstructed epidermises is carried out by hematoxylin eosin (HE) stain.

The analysis of Ki67, claudin-1, aquaporin 3 and loricrin is carried out by immunohistofluorescence.

Viewing is performed using a microscope coupled to an image analysis system. The thickness of the epidermises obtained was measured on the histological sections produced.

The rate of the various synthesized markers is proportional to the intensity of green fluorescence present on the reconstructed epidermises. A quantitative analysis of the images was carried out using an image analysis script. The results are expressed in arbitrary units (AU).

The results concerning the ability of the active substance according to the invention and of retinol to restore the thickness of the epidermis are presented in table 10.

TABLE 10

| | Measurement of the | Ability to restore |
|---|---|---|
| | thickness of the | the thickness of |
| | epidermis (μm) | the epidermis (%) |
| young reconstructed epidermises | | |
| Control | 60 | |
| older reconstructed epidermises | | |
| Control | 44 | |
| Active Substance 0.1% | 54 | +63 |
| Active Substance 0.5% | 60 | +100 |
| 10 μM Retinol | 59 | +94 |

The results concerning the ability of the active substance according to the invention to restore the synthesis of Ki-67 are presented in table 11.

TABLE 11

| | | Ability to |
|---|---|---|
| | Ki-67-positive | restore Ki-67 |
| | cells (%) | synthesis (%) |
| young reconstructed epidermises | | |
| Control | 27 | |
| older reconstructed epidermises | | |
| Control | 13 | |
| Active Substance 0.1% | 21 | +57 |
| Active Substance 0.5% | 25 | +86 |
| 10 μM Retinol | 23 | +71 |

The results concerning the ability of the active substance according to the invention to restore the synthesis of loricrin are presented in table 12.

TABLE 12

| | Synthesis | Ability to |
|---|---|---|
| | of loricrin | restore loricrin |
| | (×10⁴ AU) | synthesis (%) |
| young reconstructed epidermises | | |
| Control | 372 | |
| older reconstructed epidermises | | |
| Control | 191 | |
| Active Substance 0.1% | 256 | +36 |
| Active Substance 0.5% | 279 | +49 |
| 10 μM Retinol | 177 | 0 |

The results concerning the ability of the active substance according to the invention to restore the synthesis of claudin-1 are presented in table 13.

TABLE 13

| | Synthesis | Ability to |
|---|---|---|
| | of claudin-1 | restore claudin-1 |
| | (×10⁴ AU) | synthesis (%) |
| young reconstructed epidermises | | |
| Control | 270 | |
| older reconstructed epidermises | | |
| Control | 180 | |
| Active Substance 0.1% | 223 | +48 |
| Active Substance 0.5% | 243 | +70 |
| 10 μM Retinol | 136 | 0 |

The results concerning the ability of the active substance according to the invention to restore the synthesis of aquaporin 3 are presented in table 14.

TABLE 14

| | Aquaporin 3 | Ability to |
|---|---|---|
| | synthesis | restore aquaporin 3 |
| | (×10⁴ AU) | synthesis (%) |
| young reconstructed epidermises | | |
| Control | 355 | |
| older reconstructed epidermises | | |
| Control | 249 [symbol] | |
| Active Substance 0.1% | 308* | +56 |
| Active Substance 0.5% | 327* | +74 |
| 10 μM Retinol | 355** | +100 |

The older reconstructed epidermises have an altered barrier function, characterized by a significant reduction in the thickness of the epidermis, the synthesis of Ki-67, loricrin, claudin-1, and aquaporin 3.

Tested at 0.50% on older reconstructed epidermises, the active substance according to the invention significantly restores the thickness of the epidermis and the synthesis of:
Ki-67 of +86%;
loricrin of +49%;
claudin-1 of +70%;
aquaporin 3 of +74%.

The active substance according to the invention thus makes it possible to maintain a functional and hydrated barrier.

### Test 3: Biological Efficacy of the Active Substance According to the Invention on the Matrix Dynamics.

The objective of this study is to evaluate the ability of an active substance according to the invention to favor the production of a functional matrix array.

The extracellular dermal matrix consists mainly of a dense network of collagen I fibers. This collagen plays a key role in all phases of tissue remodeling. From 50 years of age, the quality of the dermis deteriorates gradually. The collagen fiber network is less and less dense and more and more fragmented. This effect is in particular attributable to the increase in the matrix metalloproteinase MMP-1, which degrades the collagen I and III fibers. Furthermore, hyaluronic acid is essential to maintaining the structure of the skin layers. It combines with the water and fills the intercellular spaces, which contributes to its highly hydrating power. The quantity and quality of hyaluronic acid, which is the main glycosaminoglycan in the dermis, gradually decrease with age.

The effect of the active substance according to the invention on:
the collagen I network was studied by immunocytology, on young human fibroblasts (P5) and older human fibroblasts (P22) obtained by successive replications;
the level of MMP-1 was measured by ELISA assay on human fibroblasts from donors over 60 years old and subjected to UVA exposure;
the synthesis of hyaluronic acid was evaluated by ELISA assay on human fibroblasts from donors over 60 years old

### Study of the Collagen I Network

The human fibroblasts are seeded and incubated at 37 °C in an atmosphere containing 5% CO2.

The cells are treated every other day with:
The active substance according to the invention at 0.05% and 0.10% (V/V)
a retinol solution at 0.1 μM

They are then incubated at 37 °C in an atmosphere containing 5% CO2 at 37 °C.

Immunocytological labeling of collagen I is carried out by fixing and permeabilization using primary antibody I and secondary antibody coupled to a fluorophore.

A quantitative analysis was carried out by reading the fluorescence of the various conditions tested using a fluorometer. The intensity of the collagen I network is proportional to the intensity of green fluorescence present. The results are expressed in fluorescence value (AU).

### Study of the MMP-1 Level

The older human fibroblasts are seeded and incubated at 37 °C in an atmosphere containing 5% CO2. The irradiations are carried out using a UV lamp with UVA intensities of 10 J/cm2.

The culture medium is replaced by medium containing:
The active substance according to the invention at 0.5% and 1% (V/V)
A retinol solution at 1 μM

The cells are then incubated at 37 °C in an atmosphere containing 5% CO₂ for 48 hours.

### Study of the Hyaluronic Acid Level

The older human fibroblasts are seeded and incubated at 37 °C in an atmosphere containing 5% CO2.

The culture medium is replaced by medium containing:
The active substance according to the invention at 0.5% and 1% (V/V)
A retinol solution at 1 μM

The cells are then incubated at 37 °C in an atmosphere containing 5% CO₂ for 48 hours.

The results concerning the ability of the active substance according to the invention and of retinol to restore the synthesis of the collagen I network in older fibroblasts are presented in table 15.

TABLE 15

| | | Ability to |
|---|---|---|
| | | restore the |
| | Synthesis of | synthesis of |
| | the collagen I | the collagen I |
| | network (×10³ AU) | network (%) |
| Fibroblasts | | |
| Control | 240 | |
| Older fibroblasts | | |
| Control | 177 | |
| Active Substance 0.05% | 194 | +27 |
| Active Substance 0.10% | 211 | +54 |
| 0.1 μM Retinol | 174 | |

The results concerning the effect of the active substance according to the invention on the synthesis of MMP-1 by older human fibroblasts subjected to UVA irradiation are presented in table 16.

TABLE 16

| | |
|---|---|
| | Ability to |
| MMP-1 content | reduce the MMP-1 |
| (ng/mg proteins) | level (%) |

Fibroblasts not irradiated

| | |
|---|---|
| Control | 12 |

Irradiated fibroblasts

| | | |
|---|---|---|
| Control | 63 | |
| Active Substance 0.5% | 41 | +43 |
| Active substance 1% | 40 | +45 |
| 1 μM Retinol | 46 | +33 |

The results concerning the effect of the active substance according to the invention on the synthesis of the hyaluronic acid by older human fibroblasts subjected to UVA irradiation are presented in table 17.

TABLE 17

| | |
|---|---|
| Synthesis of | Content of |
| hyaluronic acid | hyaluronic acid/ |
| (ng/mg proteins) | control (%) |

Older fibroblasts

| | | |
|---|---|---|
| Control | 3804 | |
| Active Substance 0.5% | 4678 | +23 |
| Active Substance 1% | 5066 | +33 |
| 1 μM Retinol | 4112 | +8 |

The older fibroblasts have a modified matrix array, characterized by a significant reduction in the collagen I network and the synthesis of hyaluronic acid. In response to UVA irradiations, they significantly produce MMP-1.

Tested at 0.1% of the active substance according to the invention:
significantly increases the production of the collagen I network by 54%.

Tested at 1% of the active substance according to the invention:
significantly reduces the synthesis of MMP-1 by irradiated aged fibroblasts by 45%.
significantly increases the synthesis of hyaluronic acid by 33%.

The active substance according to the invention thus maintains a functional organization of the fibers of the matrix and contributes to hydration in the dermis.

### Test 4: Cosmetic Efficacy of the Active Substance According to the Invention Evaluated in Healthy Caucasian and Asian Volunteers (In Vivo Test).

The objective of this study is to evaluate in vivo the cosmetic benefits of the active substance according to the invention formulated at 2% in emulsion form, compared to a placebo formula, on Caucasian and Asian volunteers.

The Caucasian panel is composed of 2 groups of healthy female volunteers, age 45 to 66 years having crow's feet wrinkles and having applied the placebo and the active substance according to the invention 2% on half the face twice-daily in a randomized manner for 42 days.

The cosmetic benefits of the active substance according to the invention were evaluated on the Caucasian panel at the face, after 21 and 42 days of twice-daily application, according to the following methods:
Study of the complexion radiance by visual scoring by experts (radiance, pinkness, olive color, eye fatigue)
Study of the anti-wrinkle effect by light projection.

The Asian panel is composed of 2 groups of healthy female volunteers, having crow's feet wrinkles and a dull complexion, having applied either the active substance of the invention at 2%, or the placebo, to the whole face, twice-daily for 42 days.

The cosmetic benefits of the active substance of the invention were evaluated on the Asian panel at the face, after 21 and 42 days of twice-daily application, according to the following methods:
Study of the complexion radiance by clinical scoring.
Study of the anti-wrinkle effect by clinical scoring.

### A) Effect of the Active Substance According to the Invention on Complexion Radiance

The active substance according to the invention formulated at 2% in emulsion form improves the characteristic parameters of the complexion radiance in Caucasian subjects. This effect appears substantially and significantly from 21 days.

Indeed, compared to the placebo, the active substance according to the invention:
gives a lighter and fresher complexion by significantly increasing skin radiance by 12%. This effect was observed in 89% of the volunteers and increases after 42 days of treatment (+14%) in 88% of the volunteers.
also increases the pink color significantly by 17%. This effect was observed in 61% of the volunteers and increases after 42 days of treatment (+22%) in 82% of the volunteers.
significantly reduces olive color by 8%. This effect was observed in 61% of the volunteers and intensifies after 42 days of treatment (−12%) in 88% of the volunteers.
also decreases the eye fatigue by 8%. This effect was observed in 72% of the volunteers and intensifies after 42 days of treatment (−10%) in 82% of the subjects.

The active substance according to the invention formulated at 2% in emulsion form also improves the characteristic parameters of the complexion radiance in Asian subjects. This effect appears significantly from 21 days and intensifies after 42 days of treatment.

Indeed, compared to the placebo, the active substance according to the invention:
significantly increases the skin radiance by 5%; this effect further improves after 42 days of treatment (+10%)
increases the brightness by 6%; this effect further improves after 42 days of treatment (+8%),
increases the pink color, connected to the freshness of the complexion, by 9%; this effect increases after 42 days of treatment by 18%.
causes a decrease in the olive color (−5%); this parameter further decreases after 42 days of treatment by 12%.

These effects were observed in 97% of the volunteers.

### B) Anti-Wrinkle Effect of the Active Substance According to the Invention

The active substance according to the invention formulated at 2% has a rapid and significant anti-wrinkle effect in Caucasian subjects from 21 days of twice-daily use.

Indeed, compared to the placebo, the active substance according to the invention:
smooth the skin relief of crow's feet by significantly reducing, from 21 days, the roughness of the skin (parameter Sa=−6%). This effect is maintained after 42 days of use (Sa=−7%).
reduces wrinkles by allowing a decrease in the negative volume parameter (−22% after 21 days of treatment and −19% after 42 days).

Studying the distribution of the results shows an improvement in these parameters in 83% of subjects.

Likewise, the active substance according to the invention formulated at 2% has a rapid and significant anti-wrinkle effect in Asian subjects from 21 days of twice-daily use.

Indeed, compared to the placebo, the active substance according to the invention reduces the stage of crow's feet wrinkles by 10% from 21 days of treatment. This effect intensifies after 42 days of use (decreasing the wrinkle stage by 20%). This effect was observed in 53% and 84% of the volunteers, respectively.

### Test 5: Efficacy of the Active Substance According to the Invention on the Skin Barrier in Healthy Caucasian and Asian Volunteers.

The objective of this study is to evaluate in vivo the effect of the active substance according to the invention, formulated at 2.5% in emulsion form, on the quality of the skin barrier, compared to a placebo formula on Caucasian and Asian volunteers.

The Caucasian panel is composed of two groups of 18 healthy female volunteers, age 45 to 66 years having crow's feet wrinkles and having applied the active substance according to the invention and the placebo on half the face twice-daily in a randomized manner for 42 days.

The Asian panel is composed of two groups of 32 healthy female volunteers, age 41 to 64 years, having applied either the active substance according to the invention or the placebo to the whole face morning and evening for 42 days.

Transepidermal water losses (TEWL) measurements at the cheeks were carried out using a Tewameter® before and after 21 and 42 days of twice-daily application.

From 21 days of twice-daily application and in comparison to the placebo group, the active substance according to the invention formulated at 2% in emulsion form significantly improves the quality of the skin barrier of Caucasian volunteers (17% reduction in water losses). This decrease was observed in 94% of the volunteers.

This effect is maintained after 42 days of treatment with a reduction in the TEWL equal to 15% and is observed in 82% of subjects.

From 21 days of twice-daily application to the whole face and in comparison to the placebo group, the active substance according to the invention formulated at 2% in emulsion form significantly improves the quality of the skin barrier of Asian volunteers by decreasing the transepidermal water losses by 13%. This decrease was observed in 55% of the volunteers.

This effect intensifies after 42 days of treatment with a reduction in the TEWL equal to 15% and is observed in 71% of subjects.

By reducing water losses, the active substance according to the invention tested at 2% promotes the reinforcement of the skin barrier of Caucasian and Asian volunteers.

### Test 6: Efficacy of the Active Substance According to the Invention on the Quality of the Dermal-Epidermal Junction in Healthy Caucasian Volunteers.

The objective of this study is to evaluate in vivo the effect of the active substance according to the invention formulated at 2% in emulsion form on the quality of the DEJ zone, which zone is essential to exchanges between the dermis and the epidermis, compared with a placebo formula, in Caucasian volunteers.

The Caucasian panel is composed of 2 groups of 18 healthy female volunteers, age 45 to 66 years having crow's feet wrinkles and having applied the placebo and the active substance according to the invention 2% on half the face twice-daily in a randomized manner for 42 days.

The effect of the active substance according to the invention on the quality of the dermal-epidermal junction (DEJ) was evaluated at the cheeks, compared to the placebo, after 21 and 42 days of twice-daily application, by studying the structure of the dermal-epidermal junction (LC-OCT).

In comparison to the placebo, the active substance according to the invention formulated at 2% in emulsion form promotes the sinusoidal appearance of the DEJ. This effect appears from 21 days of twice-daily application (+3%, effect observed on 78% of volunteers) and intensifies significantly after 42 days of treatment (+4%, effect observed on 88% of volunteers).

By improving the sinusoidal nature of the DEJ, the active substance according to the invention increases the surface area of the DEJ and thus promotes exchanges between the epidermis and the dermis and its involvement in the resistance to mechanical stresses.

### Efficacy of the Active Substance According to the Invention on the Quality of the Dermal Matrix in Healthy Caucasian Volunteers.

The objective of this study is to evaluate in vivo the effect of the active substance according to the invention formulated at 2% in emulsion form on the quality of the dermal matrix, compared to a placebo formula, on Caucasian volunteers.

The Caucasian panel is composed of 2 groups of 18 healthy female volunteers, age 45 to 66 years having crow's feet wrinkles and having applied the placebo and the active substance according to the invention 2% on half the face twice-daily in a randomized manner for 42 days.

The effect of the active substance according to the invention on the quality of the dermal matrix was evaluated at the cheeks, compared to the placebo, after 21 and 42 days of twice-daily application, by studying the quality of the fibers (LC-OCT).

After 21 days of twice-daily application, and in comparison with the placebo group, the active substance according to the invention, formulated at 2% in emulsion, improves the fiber quality for all of the Caucasian volunteers by 15%. This effect is maintained after 42 days of treatment (+11%) and was observed in 94% of volunteers.

By reinforcing the supporting tissue, the active substance according to the invention thus limits the slackening of the skin.

### Test 7: Comparative Test Between the Active Substance According to the Invention and a Reference Molecule: Retinol.

The objective of this study is to evaluate in vivo the cosmetic benefits of the active substance according to the invention formulated at 2% in emulsion form in Caucasian volunteers compared to a reference molecule, retinol, formulated at 0.075%.

This study was carried out on 3 groups of 18 healthy female volunteers, aged 45 to 66 years and presenting with crow's feet wrinkles. Each of the volunteers applied two of the three formulae to half the face twice-daily in a randomized manner for 42 days.

The study of the cosmetic benefits was carried out after 21 and 42 days of treatment according to the following methods:
Study of the complexion radiance by visual scoring by experts.
Study of the anti-wrinkle action by light projection.

### Study of the Complexion Radiance

The complexion radiance is evaluated by experts using the following parameters: radiance, pinkness, olive color and eye fatigue.

The results are shown in table 17.

TABLE 18

| | D 21 | | J42 | |
|---|---|---|---|---|
| | Retinol | Invention active | Retinol | Invention active |
| | 0.075% | substance at 2% | 0.075% | substance at 2% |
| Radiance | +6% | +12% | +11% | +14% |
| Pinkness | +14% | +17% | +19% | +22% |
| Olive color | −11% | −8% | −10% | −12% |
| Eye fatigue | −2% | −8% | −10% | −10% |

The active substance according to the invention has a faster effect than retinol on the radiance, pinkness and eye fatigue parameters than retinol alone from J21. This effect intensifies after 42 days of use.

### Study of the Anti-Wrinkle Effect

The anti-wrinkle effect is evaluated according to the roughness parameter Sa, evaluated on the crow's feet zone by light projection and on the negative volume, evaluated on the same zone.

The results are shown in Table 19.

TABLE 19

| | D 21 | | D 42 | |
|---|---|---|---|---|
| | Retinol | Invention active | Retinol | Invention active |
| | 0.075% | substance at 2% | 0.075% | substance at 2% |
| Parameter Sa | −5% | −6% | −7% | −7% |
| Negative | −12% | −22% | −20% | −19% |
| Volume | | | | |

In comparison to the placebo, retinol formulated at 0.075% tends to reduce the crow's feet wrinkles after 21 days of treatment (non-significant difference). It is only after 42 days of treatment that retinol begins to exhibit a significant anti-wrinkle effect (−20%).

Under the same conditions, the active substance according to the invention has a significant anti-wrinkle effect from 21 days (respectively −22%). This effect is maintained after 42 days of application (−19%).

The active substance according to the invention has a rapid anti-wrinkle effect; it allows an effect comparable to the retinol alone to be obtained.

### Test 8: Study of the Quality of the Dermal-Epidermal Junction.

The objective of this study is to evaluate in vivo the effect of the active substance according to the invention formulated at 2% in emulsion form on the quality of the DEJ zone, which is an essential zone for exchanges between the dermis and the epidermis, in Caucasian volunteers compared to a reference molecule, retinol, formulated at 0.075%.

This study was carried out on 3 groups of 18 healthy female volunteers, aged 45 to 66 years and presenting with crow's feet wrinkles. Each of the volunteers applied two of the three study formulae to half the face twice-daily in a randomized manner for 42 days.

Acquisitions carried out by LC-OCT after 21 and 42 days of treatment at the cheeks allowed evaluation of the structure of the dermal-epidermal junction.

A summary of the results corresponding to the effect of the various study formulae on the sinusoidal nature of the DEJ, studied by LC-OCT, is presented in table 20.

TABLE 20

| | D 21 | | D 42 | |
|---|---|---|---|---|
| | Retinol | Invention active | Retinol | Invention active |
| | 0.075% | substance at 2% | 0.075% | substance at 2% |
| Sinuosity | +2% | +3% | +3% | +4% |

The active substance according to the invention formulated at 2% or the retinol at 0.075% have similar results compared to the placebo.

From 21 days of application, the sinuosity of the DEJ is intensified (active substance according to the invention: +3%; retinol: +2%). This effect becomes significant after 42 days of application (active substance according to the invention: +4%; retinol: +3%).

### Test 9: Study of the Quality of the Dermal Matrix

The objective of this study is to evaluate in vivo the effect of the active substance according to the invention formulated at 2% in emulsion form on the quality of the dermal matrix in Caucasian volunteers compared to a reference molecule, retinol, formulated at 0.075%.

This study was carried out on 3 groups of 18 healthy female volunteers, aged 45 to 66 years and presenting with crow's feet wrinkles. Each of the volunteers applied two of the three study formulae to half the face twice-daily in a randomized manner for 42 days.

Acquisitions carried out by LC-OCT after 21 and 42 days of treatment at the cheeks allowed evaluation of the structure of the quality of the fibers.

The results corresponding to the effect of the various study formulae on the quality of the fibers, studied by LC-OCT, are presented in table 21.

TABLE 21

| | D 21 | | D 42 | |
|---|---|---|---|---|
| | Retinol | Invention active | Retinol | Invention active |
| | 0.075% | substance at 2% | 0.075% | substance at 2% |
| Fiber Quality | +3% | +15% | +12% | +11% |

From 21 days, compared to the placebo, the active substance according to the invention at 2% significantly improves the quality of the fibers (+15%). At this time, this improvement is not visible in the group having applied retinol at 0.075% (+3%, not significant).

After 42 days of treatment, the active substance according to the invention and the retinol exhibit comparable improvements.

Thus, the active substance according to the invention improves the quality of the fibers, in a significantly faster way than retinol (significant difference at 21 days, p=0.0020).

### Test 10: Study of the Adverse Effects.

The use of retinol is accompanied by adverse effects such as alteration of the quality of the barrier function and skin irritation phenomena.

The objective of this study is to evaluate in vivo the ability of the active substance according to the invention formulated at 2% in emulsion form and of retinol formulated at 0.075% on the unwanted effects of retinol on Caucasian volunteers.

This study was carried out on 3 groups of 18 healthy, female volunteers, aged 45 to 66 years and presenting with crow's feet wrinkles. Each of the volunteers applied two of the three formulae to half the face twice-daily in a randomized manner for 42 days.

The effects of the active substance according to the invention and of retinol were observed according to the following methods:
Study of the barrier function (Tewameter®)
Study of skin irritations (C-Cube)
Recording the skin reactions which occurred during the study

The results corresponding to the effect of the various formulae on the barrier and skin irritation are presented in table 22.

TABLE 22

| | D 21 | | D 42 | |
|---|---|---|---|---|
| | Retinol | Invention active | Retinol | Invention active |
| | 0.075% | substance at 2% | 0.075% | substance at 2% |
| Water loss | +24% | −17% | +15% | −15% |
| Parameter a* | +5% | +1% | +5% | 0% |

In comparison to the placebo, the formula containing retinol formulated at 0.075% leads to a decrease in water losses, thus preserving the quality of the skin barrier. Conversely, retinol formulated at 0.075% causes an increase in water losses (+24% at D21 and +15% at D42).

From 21 days of treatment, the application of the formula containing 0.075% of retinol significantly increases parameter a*, thus reflecting the appearance of skin redness. Compared to retinol, the active substance according to the invention at 2% does not cause the appearance of redness after 42 days of use.

Finally, in the placebo group (18 volunteers included) and in the group of the active substance according to the invention (18 volunteers included), no subject has demonstrated any adverse effects. In the retinol group (18 volunteers included), three subjects demonstrated adverse effects “likely” caused by the study product. The incident rate in this group represents 17%.

Under the conditions of this study, from 21 days of treatment, the formula containing retinol caused adverse effects.

No adverse effect was recorded with the formula containing the active substance according to the invention at 2%.

We claim:

1. A cosmetic active substance comprising at least one extract of Vaccinium macrocarpon, wherein said extract is a hydrolysate of Vaccinium macrocarpon and comprises at least 80% of peptides by weight of dry matter of the extract.

2. The cosmetic active substance according to claim 1, wherein said extract is obtained from the fruit of Vaccinium macrocarpon.

3. The cosmetic active substance according to claim 1, wherein said extract is obtained from cakes of Vaccinium Macrocarpon.

4. The cosmetic active substance according to claim 1, wherein the hydrolysate is an enzymatic hydrolysate.

5. The cosmetic active substance according to claim 1, wherein the hydrolysate comprises at least 80% of peptides, by weight of dry matter of the extract, said peptides having a size less than or equal to 2,000 Da.

6. The cosmetic active substance according to claim 5, wherein the size is between 243 and 2,000 Da.

7. The cosmetic active substance according to claim 1, wherein the peptides present in the hydrolysate comprise on average between 4 and 6 amino acids.

8. The cosmetic active substance according to claim 1, wherein said extract has a content of basic amino acid-rich cationic peptides greater than 20%.

9. The cosmetic active substance according to claim 1, wherein said extract is obtained by a method comprising the following steps:
solubilization of at least 100 g/L of Vaccinium Macrocarpon cakes in water,
subjecting the solubilized Vaccinium Macrocarpon cakes to enzymatic hydrolysis and producing an enzymatic hydrolysate, and
purifying a peptide fraction from the enzymatic hydrolysate.

10. The cosmetic active substance according to claim 1, wherein said extract being obtained by an extraction method comprising two enzymatic hydrolyses of cakes of Vaccinium Macrocarpon.

11. A cosmetic composition for topical application comprising at least one active substance according to claim 1 and a physiologically acceptable medium.

12. The cosmetic composition according to claim 11, wherein the active substance represents at least 0.1% by weight of the total weight of the composition.
